(19) United States
(12) Patent Application Publication
(10) Pub. No.: US 2005/0058768 A1
(43) Pub. Date: Mar. 17, 2005

(54) Method for coating prosthetic stents

(76) Inventor: Eyal Teichman, Hod-Hasharon (IL)

Correspondence Address: KUDIRKA & JOBSE, LLP, ONE STATE STREET, SUITE 800, BOSTON, MA, 02109, US

(21) Appl. No.: 10/662,375

(22) Filed: Sep. 16, 2003

(51) Int. Cl. A61L 2/00

(52) U.S. Cl. 427/2.24

(57) ABSTRACT

A method and device for coating stents by uniformly encapsulating the struts of the stent. The stent is not contacted by the applicator, and the coating material is allowed to flow around the stent strut for full encapsulation, even when application of coating material occurs on only one surface of the stent.

### FIELD OF THE INVENTION

The present invention relates to the medical field of coating prosthetic stents with coating materials, in particular methods for providing coatings for the exterior surface and interior surface of the stent at the manufacturing stage.

### BACKGROUND OF THE INVENTION

The practice of coating implantable prosthetic stents with a synthetic or biological active or inactive agent is known. Numerous processes have been proposed for the application of such a coating. Soaking or dipping the implantable device in a bath of liquid medication is suggested by U.S. Pat. No. 5,922,393 to Jayaraman, soaking in an agitated bath, U.S. Pat. No. 6,129,658 to Delfino et al. Devices introducing heat and/or ultrasonic energy in conjunction with the medicated bath are disclosed in U.S. Pat. Nos. 5,891,507 to Jayaraman and 6,245,4 BI to Alt. The device of U.S. Pat. No. 6,214,1 BI to Taylor et al. suggest spraying the medication by way of pressurized nozzles.

Coating can be applied at the time of manufacture. However, existing methods deposit coating material on all surfaces of the stent such that excess coating material is needed. This results in an uncontrolled coating thickness and the potential for coating material covering portions of the interstices of the stent (so-called “webbing”).

Additionally, poor control of the coating process in the prior art requires inefficient expenditure of the coating material.

### SUMMARY OF THE INVENTION

In accordance with the invention, a device and method allow the exterior surface and the interior surface of a stent to be coated, while minimizing the amount of coating material necessary to cover these surfaces, and while maintaining desired distributions of coating material on each surface.

In one illustrative embodiment, the present invention includes devices and apparatus utilizing drop-on-demand or spray exterior surface and interior surface applicators which coat the exterior surface and interior surface of the stent. The stent is may be mounted by gripping it to a stent rotation mechanism. The applicators are guided by software which processes information from a detector which scans the stent. The device uses a control scheme to coat the desired portions of the exterior surface and interior surface of the stent.

The struts of a stent, which are typically metallic and rectangular in cross section, are coated on all sides by the calculated application of coating material on one or two sides (e.g., the interior and exterior) of the strut, while the remaining two (e.g., circumferentially laterally oriented) sides are coated by the flowing of coating material around the strut, assisted perhaps by the coherence created by surface tension in the fluent coating material. Of course, a stent may have struts having a non-rectangular cross section, for instance square, round or oval. Although the illustrative embodiment of the instant invention is described with reference to a stent having rectangular cross section struts, the coating of stents having other cross sectional shapes is equally advantageously achieved by the present invention.

In a further feature of the illustrative embodiment, the device comprises drop-on-demand exterior surface and interior surface applicators which coat either or both the exterior surface and interior surface of the stent. The stent can be mounted, for example, by sliding it into an external tube and over an internal cylinder. The applicators are guided by software which processes information from a detector which scans the stent. The control scheme of the invention permits coating the desired portions of either or both the exterior surface and interior surface of the stent. The method comprises either sequentially coating the exterior and interior surfaces of the stent, and allowing the fluent coating material to flow to the remaining sides of a stent strut, or coating either the exterior or interior surface and allowing the coating material to flow around the strut. Two or more applicators can be used on a single side of the strut.

In another embodiment, an illustrative device can comprise drop-on-demand exterior surface and interior surface applicators which coat the exterior surface and interior surface of the stent. The applicators are guided by software which processes information from a detector which scans the stent. The device uses a control scheme to coat the desired portions of the exterior surface and interior surface of the stent.

In the illustrative embodiments described herein, the scanning of the stent can occur prior to coating (pre-scanning) or in conjunction with coating (“real-time” scanning). Pre-scanning provides information collected by the detector to determine coating coordinates for the applicators during the coating process. “Real-time” scanning provides “real-time” information collected by the detector to control the dispensing of coating material by the applicator. In some embodiments, the detector can be mechanically connected to the applicator such that the movement of the detector follows the movement of the applicator. Additionally, scanning the stent after coating (post-scanning) can determine whether the coating material has been applied properly to the stent surfaces.

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate several embodiments of the invention and together with the description, serve to explain the principles of the invention.

FIG. 1 is a view of an exemplary stent coating device having interior and exterior surface applicatiors.

FIG. 2 is a view of the exemplary stent coating device of FIG. 1 depicting a magnified view of a drop on demand (DOD) applicator.

FIG. 3 is a view of an exemplary stent coating device of FIG. 1 depicting an interior surface applicator.

FIG. 4 is a view of an exemplary stent coating device of FIG. 1 depicting an exterior surface applicator.

FIG. 5 is a schematic representation of an illustrative embodiment of a stent coating device according to the present invention.

FIG. 6 is a schematic representation of an illustrative embodiment of a stent coating method according to the present invention.

FIG. 7 is a schematic representation of an illustrative embodiment of a two-sided stent coating method according to the present invention.

FIG. 8 is a schematic representation of an illustrative embodiment of a one-sided stent coating method according to the present invention.

FIG. 9 is a schematic representation of an illustrative embodiment of a one sided, double-shot stent coating method according to the present invention.

### DESCRIPTION OF THE EMBODIMENTS

Reference will now be made in detail to the present illustrative embodiments of the invention, examples of which are illustrated in the accompanying drawings. Wherever possible, the same reference numbers will be used throughout the drawings to refer to the same or like parts.

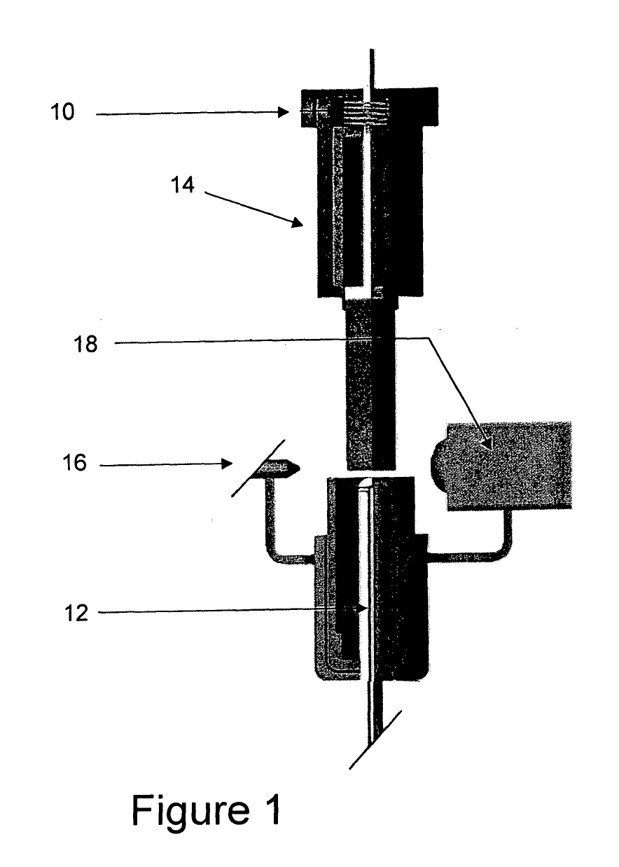

FIG. 1 illustrates an embodiment 10 of a stent coating device. The interior surface applicator 12 is located within the bottom assembly of the stent rotation mechanism 14. The exterior surface applicator 16 and detector 18 are located adjacent to the bottom assembly of the stent rotation mechanism 14.

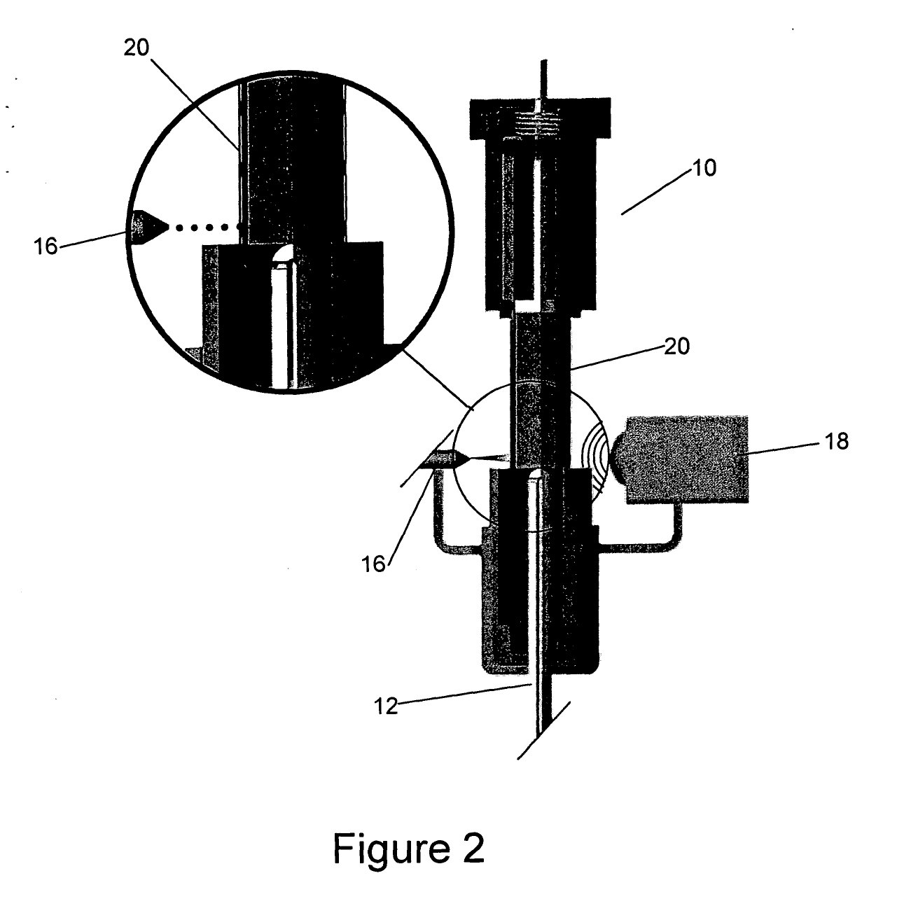

FIG. 2 illustrates a further view of the embodiment 10 of a device for coating the stent. The stent coating mechanism can rotate the stent for coating while the applicators translate. As above, the device can include, and the stent 10 can be coated by, an exterior surface applicator 16 and/or an interior surface applicator 12. The exterior surface applicator 16 and interior surface applicator 12 can move in a relative circumferential or a spiral motion, in synchrony or in sequence. The exterior surface applicator can be staggered with interior surface applicator 12 such that the exterior surface applicator dispenses coating material on the exterior surface of the stent, followed by the interior surface applicator 12 dispensing coating material on the interior surface of the stent. Alternatively a single applicator, either interior or exterior can be used. A detector 18 can scan the stent as it is rotated and the data used to calculate movement of the stent 20, and/or the applicator(s) 12, 16.

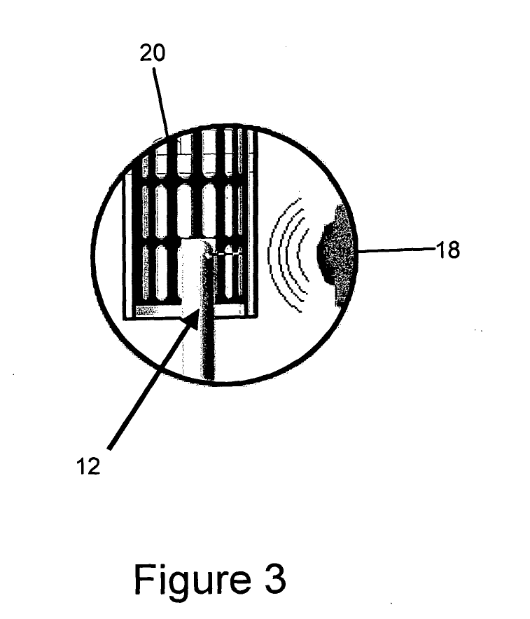

FIG. 3 illustrates an embodiment of a method of coating the interior surface of the stent 20. The interior surface applicator 12 is positioned inside the stent 20 and can be moved along the axis of the stent 20, or the stent 20 can be moved relative to the applicator 12. The applicator 12 can rotate, or the stent 20 can rotate. Relative rotation and translation can occur simultaneously. The illustrative embodiment of an interior surface applicator 12 can be a very thin profile applicator with a diameter of less than 1.8 mm.

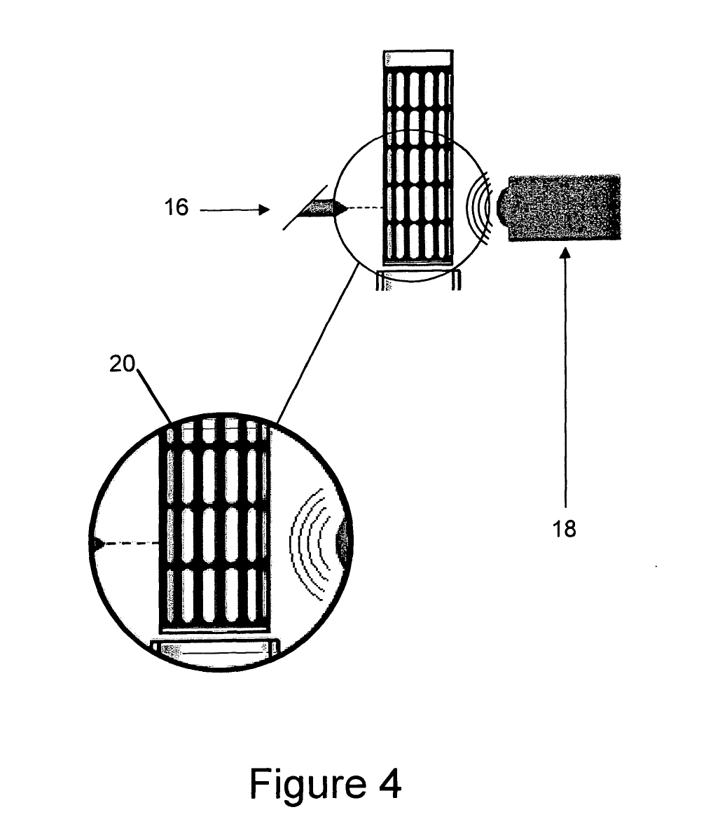

FIG. 4 depicts an illustrative embodiment of a method of coating the external surface of a stent 20. The stent 20 can be rotated as the exterior surface applicator 16 dispenses coating material on the exterior surface of the stent 20. The detector 18 scans the stent as the stent rotates.

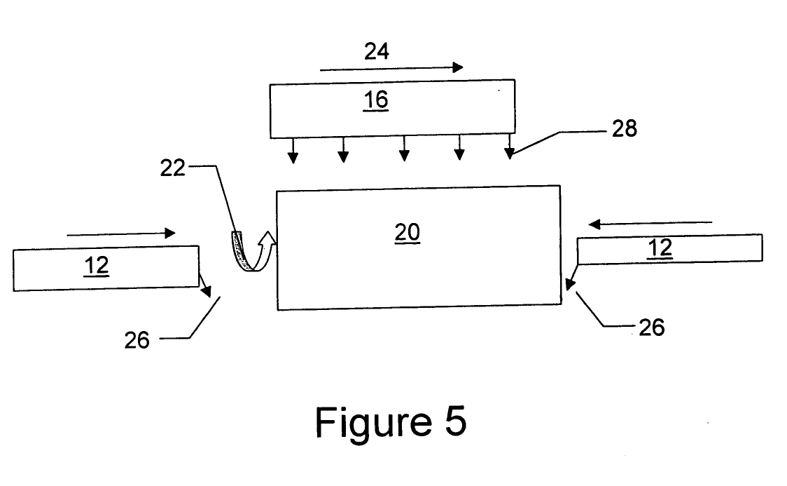

FIG. 5 schematically illustrates an embodiment of a stent coating device with applicators for the exterior surface 16 and interior surface 12 of the stent. The stent 20 is rotated during scanning and during coating. The exterior surface of the stent is coated by the exterior surface applicator 16. In one embodiment, a detector 18 can be mounted with the exterior surface applicator 16 so that it can be positioned by the same drive mechanism as the applicator. Alternatively, a detector 18 can be separately mounted and positioned. The interior surface applicator 12 is positioned inside the stent 20 to coat the interior surface. In one embodiment, two interior surface applicators 12, as shown in the figure are introduced from each end of the stent 20. The interior surface applicators 12 are each positioned by an interior surface applicator 12 drive mechanism.

In one embodiment, the stent is scanned by a detector. Software in the CPU of the device (not shown in the FIG.) analyze the information from the detector to determine the coating coordinates for the exterior surface and the interior surface of the stent. In another embodiment, the CPU is uploaded with information from a previous scan of the stent or coating coordinates specified by the stent manufacturer. The coating coordinates can be transferred to a servo controller. In one illustrative embodiment, the applicators are drop on demand (DOD) jets. Arrows 26 show internal drop direction, while arrows 28 show external drop directions. These directions are adjusted, along with drop size and velocity, as well as applicator placement, to optimize encapsulation.

In one embodiment, the coating material is applied in a spiral with the rotation of the stent and lateral movement of the exterior surface applicator drive mechanisms. The relative motion between the ink jets and the stent surface is combined with lateral (linear) and rotational components to the motion. This type of movement allows on-the-fly raster type coating. When the coating process is complete, the interior surface applicators 12 are withdrawn from the two ends of the stent and the stent 20 is unloaded from the device. In one embodiment, the stent is scanned by the detector prior to the removal from the device. The information from this scan allows the CPU to inspect the coating material and provide quality assurance that the desired coating was applied.

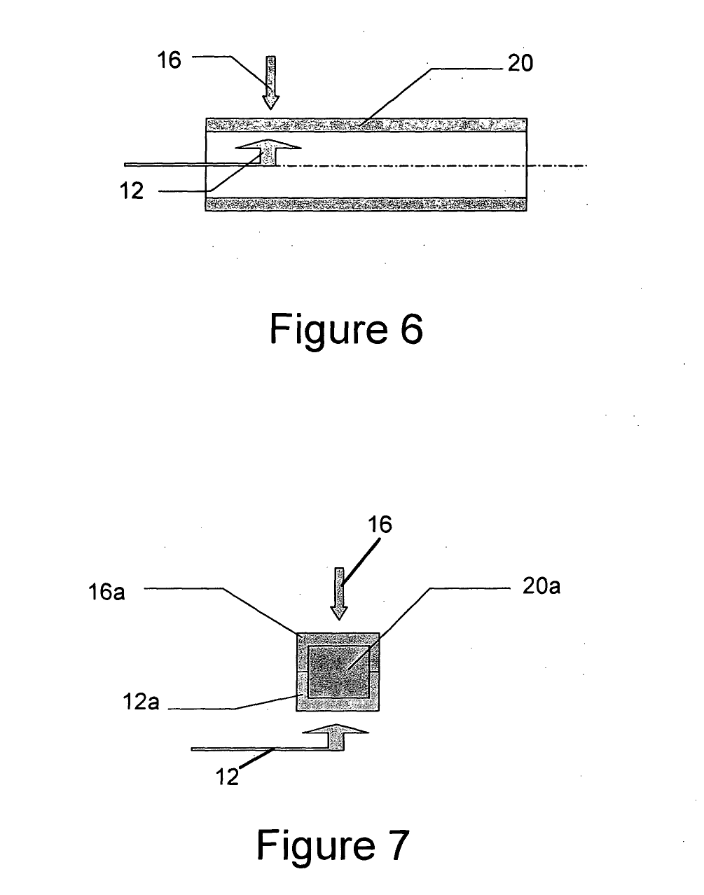

FIG. 6 schematically depicts an internal jet 12 and an external jet 16 in relation to a stent 20. FIG. 7 depicts the cross section 20a of a stent strut, and the resulting coating 16a applied by the external jet 16 and the coating 12a applied by the internal jet 12. This illustrative embodiment of the present invention advantageously applies the coating to the interior and exterior surfaces, and allows the coating material to flow around the entire external perimeter of the stent strut.

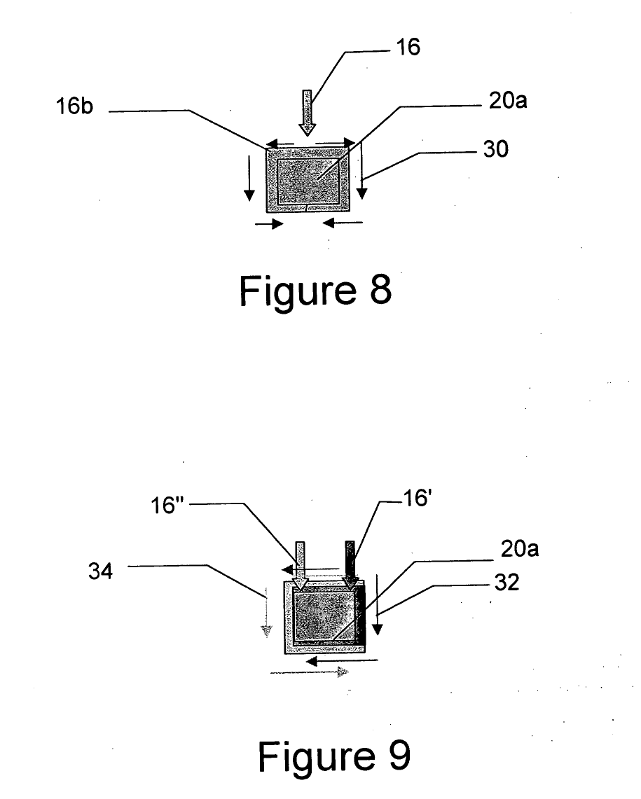

FIG. 8 schematically depicts the cross section 20a of a stent strut, and the resulting coating 16b applied by only an external jet 16. This illustrative embodiment of the present invention advantageously applies the coating to the exterior surface, and allows the coating material to flow as shown by arrows 30 around the entire external perimeter of the stent strut. Of course, a similar approach can be taken using only an interior jet.

FIG. 9 schematically depicts the cross section 20a of a stent strut, and the resulting coating applied by a pair of external jets 16′, 16″. This illustrative embodiment of the present invention advantageously applies the coating to the exterior surface, and allows the coating material to flow around the entire external perimeter of the stent strut. As depicted, the flow from one external jet 16′ flows more predominantly to one side of the strut in the direction of arrows 32, while the flow from the other external jet 16″ flows more predominantly to the other side of the strut in the direction of arrows 34. Of course, a similar approach can be taken using interior jets. Alternatively, a single jet can be used to sequentially coat first proximate one side at position 16′, then the other at position 16″.

In all illustrative embodiments, the fluent coating material tends toward a uniform coating. This perhaps can be attributed to the effects of surface tension. Advantageously, surface tension may also tends to adhere the coating to the surface of the stent substrate.

In one illustrative embodiment, the interior surface applicator 12 is a pipette such as the MicroDrop®) microdosing system as described in http://www.microdrop.de/html/microdropprod.html by MicroDrop GmbH of Norderstedt, Germany. The MicroDrop® dispenser heads are designed to process a wide range of different liquids. For example, coating materials with viscosities between 0.5 mPas to 20 mPas can be dispensed. For coating materials with higher viscosities, the MicroDrop® dispenser provides a nozzle heater to keep the coating material temperature constant to avoid viscosity changes by variations of ambient temperature. Such temperature control is desirable in the range of 20 mPas to 100 mPas. For coating materials with viscosities above 100 mPas it is desirable to reduce the liquid viscosity below 100 mPas. For this purpose, the MicroDrop® dispenser is equipped with a nozzle heater which is controlled by a thermo sensor. The temperature controller in the drive electronics maintains a temperature variation of less than 1° C. The MicroDrop® dispenser head can be arranged in any direction, i.e. both horizontal or vertical with respect to its reservoir of coating material. The the MicroDrop® dispenser can also be connected to a strobe diode detector to obtain information in the form of still images of the droplet emissions.

Coating materials are any liquid or semi-liquid material chosen from polymers, therapeutic agents, and thin films. The coating materials which can be used in conjunction with the present invention are any desired, suitable substances. In some embodiments, the coating materials comprise therapeutic agents, applied to the medical devices alone or in combination with solvents in which the therapeutic agents are at least partially soluble or dispersible or emulsified, and/or in combination with polymeric materials as solutions, dispersions, suspensions, latices, etc. The terms “therapeutic agents” and “drugs” are used interchangeably herein and include pharmaceutically active compounds, nucleic acids with and without carrier vectors such as lipids, compacting agents (such as histones), virus, polymers, proteins, and the like, with or without targeting sequences. The coating may provide for controlled release, which includes long-term or sustained release, of a bioactive material. Specific examples of therapeutic or bioactive agents used in conjunction with the present invention include, for example, pharmaceutically active compounds, proteins, oligonucleotides, ribozymes, anti-sense genes, DNA compacting agents, gene/vector systems (i.e., anything that allows for the uptake and expression of nucleic acids), nucleic acids (including, for example, recombinant nucleic acids; naked DNA, cDNA, RNA; genomic DNA, cDNA or RNA in a non-infectious vector or in a viral vector which may have attached peptide targeting sequences; antisense nucleic acid (RNA or DNA); and DNA chimeras which include gene sequences and encoding for ferry proteins such as membrane translocating sequences (“MTS”) and herpes simplex virus-1 (“VP22”)), and viral, liposomes and cationic polymers that are selected from a number of types depending on the desired application. For example, biologically active solutes include anti-thrombogenic agents such as heparin, heparin derivatives, urokinase, and PPACK (dextrophenylalanine proline arginine chloromethylketone); prostaglandins, prostacyclins/prostacyclin analogs; antioxidants such as probucol and retinoic acid; angiogenic and anti-angiogenic agents; agents blocking smooth muscle cell proliferation such as rapamycin, angiopeptin, and monoclonal antibodies capable of blocking smooth muscle cell proliferation; anti-inflammatory agents such as dexamethasone, prednisolone, corticosterone, budesonide, estrogen, sulfasalazine, acetyl salicylic acid, and mesalamine, lipoxygenase inhibitors; calcium entry blockers such as verapamil, diltiazem and nifedipine; antineoplastic/antiproliferative/anti-mitotic agents such as paclitaxel, 5-fluorouracil, methotrexate, doxorubicin, daunorubicin, cyclosporine, cisplatin, vinblastine, vincristine, colchicine, epothilones, endostatin, angiostatin, Squalamine, and thymidine kinase inhibitors; L-arginine; antimicrobials such astriclosan, cephalosporins, aminoglycosides, and nitorfuirantoin; anesthetic agents such as lidocaine, bupivacaine, and ropivacaine; nitric oxide (NO) donors such as lisidomine, molsidomine, NO-protein adducts, NO-polysaccharide adducts, polymeric or oligomeric NO adducts or chemical complexes; anti-coagulants such as D-Phe-Pro-Arg chloromethyl ketone, an RGD peptide-containing compound, heparin, antithrombin compounds, platelet receptor antagonists, anti-thrombin antibodies, anti-platelet receptor antibodies, enoxaparin, hirudin, Warafin sodium, Dicumarol, aspirin, prostaglandin inhibitors, platelet inhibitors and tick antiplatelet factors; interleukins, interferons, and free radical scavengers; vascular cell growth promoters such as growth factors, growth factor receptor antagonists, transcriptional activators, and translational promotors; vascular cell growth inhibitors such as growth factor inhibitors (e.g., PDGF inhibitor—Trapidil), growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; Tyrosine kinase inhibitors, chymase inhibitors, e.g., Tranilast, ACE inhibitors, e.g., Enalapril, MMP inhibitors, (e.g., IIomastat, Metastat), GP IIb/IIIa inhibitors (e.g., Intergrilin, abciximab), seratonin antagnonist, and 5-HT uptake inhibitors; cholesterol-lowering agents; vasodilating agents; agents which interfere with endogeneus vascoactive mechanisms; survival genes which protect against cell death, such as anti-apoptotic Bcl-2 family factors and Akt kinase; and combinations thereof; and beta blockers. These and other compounds may be added to a coating solution, including a coating solution that includes a polymer, using similar methods and routinely tested as set forth in the specification. Any modifications are routinely made by one skilled in the art. Polynucleotide sequences useful in practice of the invention include DNA or RNA sequences having a therapeutic effect after being taken up by a cell. Examples of therapeutic polynucleotides include anti-sense DNA and RNA; DNA coding for an anti-sense RNA; or DNA coding for tRNA or rRNA to replace defective or deficient endogenous molecules. The polynucleotides of the invention can also code for therapeutic proteins or polypeptides. A polypeptide is understood to be any translation product of a polynucleotide regardless of size, and whether glycosylated or not. Therapeutic proteins and polypeptides include as a primary example, those proteins or polypeptides that can compensate for defective or deficient species in an animal, or those that act through toxic effects to limit or remove harmful cells from the body. In addition, the polypeptides or proteins that can be incorporated into the polymer coating, or whose DNA can be incorporated, include without limitation, angiogenic factors and other molecules competent to induce angiogenesis, including acidic and basic fibroblast growth factors, vascular endothelial growth factor, hif-1, epidermal growth factor, transforming growth factor alpha. and .beta., platelet-derived endothelial growth factor, platelet-derived growth factor, tumor necrosis factor alpha., hepatocyte growth factor and insulin like growth factor; growth factors; cell cycle inhibitors including CDK inhibitors; anti-restenosis agents, including p15, p16, p18, p19, p21, p27, p53, p57, Rb, nFkB and E2F decoys, thymidine kinase (“TK”) and combinations thereof and other agents useful for interfering with cell proliferation, including agents for treating malignancies; and combinations thereof. Still other useful factors, which can be provided as polypeptides or as DNA encoding these polypeptides, include monocyte chemoattractant protein (“MCP-1”), and the family of bone morphogenic proteins (“BMP's”). The known proteins include BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15, and BMP-16. Currently preferred BMPs are any of BMP-2, BMP-3, BMP-4, BMP-5, BMP-6 and BMP-7. These dimeric proteins can be provided as homodimers, heterodimers, or combinations thereof, alone or together with other molecules. Alternatively, or in addition, molecules capable of inducing anupstream or downstream effect of a BMP can be provided. Such molecules include any of the “hedgehog” proteins, or the DNA's encoding them. Coating materials other than therapeutic agents include, for example, polymeric materials, sugars, waxes, and fats, applied alone or in combination with therapeutic agents, and monomers that are cross-linked or polymerized. Such coating materials are applied in the form of, for example, powders, solutions, dispersions, suspensions, and/or emulsions of one or more polymers, optionally in aqueous and/or organic solvents and combinations thereof or optionally as liquid melts including no solvents. When used with therapeutic agents, the polymeric materials are optionally applied simultaneously with, or in sequence to (either before or after), the therapeutic agents. Such polymeric materials employed as, for example, primer layers for enhancing subsequent coating applications (e.g., application of alkanethiols or sulfhydryl-group containing coating solutions to gold-plated devices to enhance adhesion of subsequent layers), layers to control the release of therapeutic agents (e.g., barrier diffusion polymers to sustain the release of therapeutic agents, such as hydrophobic polymers; thermal responsive polymers; pH-responsive polymers such as cellulose acetate phthalate or acrylate-based polymers, hydroxypropyl methylcellulose phthalate, and polyvinyl acetate phthalate), protective layers for underlying drug layers (e.g., impermeable sealant polymers such as ethylcellulose), biodegradable layers, biocompatible layers (e.g., layers comprising albumin or heparin as blood compatible biopolymers, with or without other hydrophilic biocompatible materials of synthetic or natural origin such as dextrans, cyclodextrins, polyethylene oxide, and polyvinyl pyrrolidone), layers to facilitate device delivery (e.g., hydrophilic polymers, such as polyvinyl pyrrolidone, polyvinyl alcohol, polyalkylene gylcol (i.e., for example, polyethylene glycol), or acrylate-based polymer/copolymer compositions to provide lubricious hydrophilic surfaces), drug matrix layers (i.e., layers that adhere to the medical device and have therapeutic agent incorporated therein or thereon for subsequent release into the body), and epoxies. When used as a drug matrix layer for localized drug delivery, the polymer coatings of the present invention comprise any material capable of absorbing, adsorbing, entrapping, or otherwise holding the therapeutic agent to be delivered. The material is, for example, hydrophilic, hydrophobic, and/or biodegradable, and is preferably selected from the group consisting of polycarboxylic acids, cellulosic polymers, gelatin, polyvinylpyrrolidone, maleic anhydride polymers, polyamides, polyvinyl alcohols, polyethylene oxides, glycosaminoglycans, polysaccharides, polyesters, polyurethanes, silicones, polyurea, polyacrylate, polyacrylic acid and copolymers, polyorthoesters, polyanhydrides such as maleic anhydride, polycarbonates, polyethylene, polypropylenes, polylatic acids, polystyrene, natural and synthetic rubbers and elastomers such as polyisobutylene, polyisoprene, polybutadiene, including elastomeric copolymers, such as Kraton®., styrene-isobutylene-styrene (SIBS) copolymers; polyglycolic acids, polycaprolactones, polyhydroxybutyrate valerates, polyacrylamides, polyethers, polysaccharides such as cellulose, starch, dextran and alginates; polypeptides and proteins including gelatin, collagen, albumin, fibrin; copolymers of vinyl monomers such as ethylene vinyl acetate (EVA), polyvinyl ethers, polyvinyl aromatics; other materials such as cyclodextrins, hyaluronic acid and phosphorylcholines; and mixtures and copolymers thereof. Coatings from polymer dispersions such as polyurethane dispersions (BAYHDROL, etc.) and acrylic latex dispersions are also within the scope of the present invention. Polymers can include polyurethanes; polyacrylic acid, and aqueous coating compositions comprising an aqueous dispersion or emulsion of a polymer having organic acid functional groups and a polyfunctional crosslinking agent having functional groups capable of reacting with organic acid groups. The release rate of drugs from drug matrix layers is largely controlled, for example, by variations in the polymer structure and formulation, the diffusion coefficient of the matrix, the solvent composition, the ratio of drug to polymer, potential chemical reactions and interactions between drug and polymer, the thickness of the drug adhesion layers and any barrier layers, and the process parameters, e.g., drying, etc. The coating(s) applied by the methods and apparatuses of the present invention may allow for a controlled release rate of a coating substance with the controlled release rate including both long-term and/or sustained release. Additionally, a coating substance may include suspension particles, e.g., a powder. For example, the suspension particles may be fused to the surface of the prosthesis by a coating solution.

The coatings of the present invention are applied such that they result in a suitable thickness, depending on the coating material and the purpose for which the coating(s) is applied. As an example, coatings applied for localized drug delivery are typically applied to a thickness of 1 to 30 microns, or 2 to 20 microns. Very thin coatings, e.g., of about 100 angstroms., and much thicker coatings, e.g., more than 30 microns, are also possible. It is also within the scope of the present invention to apply multiple layers of the same or different coating materials, which may perform identical or different functions (e.g., to provide for biocompatibility, to control drug release, etc.).

Using drop on demand (DOD) jets that are driven by stent pre-scan data solves the problems of the prior art, and mitigates the loss of coating material during the coating process. Encapsulation is achieved by pre-tuning the amount of material to very accurate places of over the stent surface, and aiming the jets that will make allow each drop's volume to flow to cover a plurality of surfaces of the stent's struts.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1-8. Canceled

9. A method for coating a stent comprising:
providing a stent having a substantially cylindrical shape with an interior and an exterior, said stent comprising at least one strut having regions with exposed strut surfaces disposed around a periphery of said strut;
positioning said stent and at least one applicator relative to one another in spaced apart relation;
adjusting at least one application parameter of the at least one applicator; and
dispensing a coating from said at least one applicator, in accordance with the adjusted at least one application parameter, onto said at least one strut of said stent such that said coating material flows over said exposed strut surfaces and the periphery to form a substantially uniform coating on said exposed strut surfaces.

10. The method of claim 9, wherein said step of positioning at least one applicator comprises positioning one applicator on an exterior aspect of a substantially cylindrical stent.

11. The device of claim 9, wherein said step of positioning at least one applicator comprises positioning two applicators disposed on an exterior aspect of a substantially

12. The device of claim 9, wherein said step of positioning at least one applicator comprises positioning one applicator disposed on an interior aspect of a substantially cylindrical stent.

13. The method of claim 9, wherein said step of positioning at least one applicator comprises positioning two applicators disposed on an interior aspect of a substantially cylindrical stent.

14. The method of claim 9, wherein said step of positioning at least one applicator comprises positioning at least one applicator disposed on an exterior aspect and at least one applicator disposed on an interior aspect of a substantially cylindrical stent.

15. The method of claim 17, further comprising the step of sensing the stent topography, and using said topography in said positioning step.

16. The method of claim 17, wherein said dispensing step further comprises dispensing material from a drop on demand (DOD) jet.

17. The method of claim 10, wherein said positioning step further comprises the step of targeting a center of a strut outer surface, and
said adjusting step further comprises adjusting drop size and drop velocity parameters of said at least one applicator so as to achieve full strut encapsulation of said strut by said dispensing step.

18. The method of claim 10, wherein said positioning step further comprising the step of targeting first one side, then another side, of a strut outer surface,
said dispensing step comprises the step of dispensing first at one side, then another side, of a strut outer surface, and
adjusting drop size, drop velocity and drop location parameters of said applicator so as to achieve full strut encapsulation.

19. The method of claim 14, wherein said positioning step further comprising the step of targeting the center of a strut outer surface and a strut inner surface, and
adjusting drop size and drop velocity parameters of said applicator so as to achieve full strut encapsulation by said dispensing step.
